SEQUENCE LISTING (8 sequences)

SEQ ID NO: 1 - DNA, 36 bp, Artificial Sequence
  Mutagenesis primer for HIF-1alpha P564A
cttggagatgttagctgcctatatcccaatggatga

SEQ ID NO: 2 - DNA, 36 bp, Artificial Sequence
  Mutagenesis primer for HIF-1alpha P564A
tcatccattgggatataggcagctaacatctccaag

SEQ ID NO: 3 - DNA, 24 bp, Artificial Sequence
  Mutagenesis primer for HIF-1alpha P402A
ctttgctggccgcagccgctggag

SEQ ID NO: 4 - DNA, 24 bp, Artificial Sequence
  Mutagenesis primer for HIF-1alpha P402A
ctccagcggctgcggccagcaaag

SEQ ID NO: 5 - DNA, 20 bp, Artificial Sequence
  NEDL1 primer
ccagggaaccaaagcataga

SEQ ID NO: 6 - DNA, 20 bp, Artificial Sequence
  NEDL1 primer
gtggatgcggatgtagtgcg

SEQ ID NO: 7 - DNA, 30 bp, Artificial Sequence
  beta-Actin primer
tgacggggtcacccacactgtgcccatcta

SEQ ID NO: 8 - DNA, 30 bp, Artificial Sequence
  beta-Actin primer
ctagaagcatttgcggtggacgatggaggg

(19) United States
(12) Patent Application Publication
(10) Pub. No.: US 2009/0269794 A1
(43) Pub. Date: Oct. 29, 2009

(54) SCREENING METHOD FOR COMPOUND INHIBITING DEVELOPMENT OR PROGRESSION OF FAMILIAL AMYOTROPHIC LATERAL SCLEROSIS AND DIAGNOSTIC METHOD FOR FAMILIAL AMYOTROPHIC LATERAL SCLEROSIS

(75) Inventors: Akira Nakagawara, Chiba-shi (JP); Keizo Takenaga, Chiba-shi (JP); Shigenobu Mochizuki, Chiba-shi (JP)

Correspondence Address: SUGHRUE MION, PLLC, 2100 PENNSYLVANIA AVENUE, N.W., SUITE 800, WASHINGTON, DC, 20037, US

(73) Assignees: HISAMITSU PHARMACEUTICAL CO., INC., Tosu-shi, Saga (JP); CHIBA-PREFECTURE, CHIBA-SHI, CHIBA (JP)

(21) Appl. No.: 12/063,347

(22) PCT Filed: Aug. 9, 2006
(86) PCT No.: PCT/JP2006/315736

§ 371 Date: Jul. 22, 2008

(30) Foreign Application Priority Data
Aug. 11, 2005 (JP) 2005-233435

(51) Int. Cl. C12Q 1/02

(52) U.S. Cl. 435/29

(57) ABSTRACT

A screening method comprising the following steps is provided.
A step of culturing cells in the presence and in the absence of a test compound,
a step of measuring HIF-1α expression in the respective cultured cells, and
a step of determining the test compound to be a compound that inhibits onset or progression of familial amyotrophic lateral sclerosis if the HIF-1α expression in the cells cultured in the presence of the test compound is less than the HIF-1α expression in the cells cultured in the absence of the test compound.

### TECHNICAL FIELD

The present invention relates to a method for screening of compounds that inhibit onset or progression of familial amyotrophic lateral sclerosis. The invention further relates to a method for diagnosis of familial amyotrophic lateral sclerosis.

### BACKGROUND ART

Amyotrophic Lateral Sclerosis (ALS) is an intractable neurological disease wherein deficit of motor neurons in the spine leads to walking difficulty within several years of onset and eventually to death. The cause of ALS is not understood and currently no method of prevention or treatment exists. As a result of positional cloning experiments, it was reported in 1993 that approximately 20% of autosomal dominantly inherited Familial ALS (Familial Amyotrophic Lateral Sclerosis, or FALS) cases occur due to mutation in the gene superoxide dismutase-1 (SOD-1) (Non-patent document 1). SOD-1 is an enzyme that catalyzes the reaction of reactive oxygen species (ROS) in the cytoplasm to hydrogen peroxide. However, since no correlation has been found between SOD-1 activity levels and progression of FALS (Non-patent document 2), it is believed that toxic-gain-of-function by mutant SOD-1 may be a cause of FALS, and determination of this cause has become a major focus of attention.

While no correlation has been found between SOD-1 activity levels and progression of FALS, it has been reported that excess ROS are produced in motor neurons by the abnormal oxidation-reduction system, suggesting their involvement in onset of FALS (Non-patent documents 3 and 4). Moreover, research indicates that antioxidants that inhibit ROS production may delay onset of ALS (Non-patent documents 5 and 6).

It has been reported that the spinal cords of FALS model mice contain high levels of the inflammatory cytokines tumor necrosis factor-α (TNFα) and interleukin-1 (IL-1) prior to onset, suggesting that the inflammatory response also plays a role in motor neuron apoptosis (Non-patent documents 7 and 8).

The HECT E3 ligase NEDL1, which binds with and ubiquitinates mutant SOD-1, has recently been discovered (Non-patent document 9). NEDL1 ubiquitinates mutant SOD-1 and promotes its degradation in the proteasome, but does not ubiquitinate wild-type SOD-1. In addition, a positive correlation has been found between the severity of FALS associated with several different types of mutant SOD-1, and the extent of binding between each mutant SOD-1 and NEDL1. Mutant SOD-1 was known to bind with translocon-associated protein-δ (TRAP-δ), one of the components of the hydrophilic channel (translocon) on the rough endoplasmic reticulum, but research showed that NEDL1 also binds with TRAP-δ. It was also demonstrated that NEDL1 binds with and ubiquitinates Dishevelled-1 (DVL1), a key molecule in the Wnt signaling, as well. From these results, it is speculated that NEDL1 forms ubiquitinated complexes with mutant SOD, TRAP-δ and Dv11 and that motor neurons may die due to the toxicity of the complexes.

It is known, as well, that cells exposed to hypoxia conditions produce hypoxia-inducible factor-1 (HIF-1), a key molecule in the hypoxia response signaling pathway (Non-patent document 10). HIF-1 is composed of two subunits, HIF-1α and HIF1-β/ARNT (aryl hydrocarbon receptor nuclear translocator), and both subunits belong to the subfamily of basic helix-loop-helix (bHLH) transcription factors that possess a PAS domain (Non-patent document 11). The HIF-1α molecule has two transcription activation regions. HIF-1α is continuously expressed on the mRNA level, but the protein is degraded as soon as it is synthesized and is normally below the detection limit. In contrast, HIF-1β is constitutively expressed. HIF Prolyl hydroxylase (PDH) is involved in degradation of HIF-1α, and four PDHs (PDH1-4) have been discovered as of the current writing. PDH hydroxylates the 402nd and 564th proline residues of HIF-1α protein. Proline-hydroxylated HIF-1α is ubiquitinated by E3-ubiquitin ligase complex comprising von Hippel Lindau protein (pVHL), elongin B, elongin C, CUL and RBX1, and promptly degraded by the 26S proteasome. Under hypoxia conditions, however, oxygen molecule-requiring PDH activity is reduced and HIF-1α is no longer degraded, so that it becomes detectable as protein. When cells are exposed to desferrioxamine (DFO) which has PHD inhibitory action, HIF-1α is detected even with normal oxygen conditions (Non-patent document 12). HIF-1α having the 402nd and 564th prolines substituted with alanine (HIF-1α^{P402A/P564A}) is not hydroxylated by PDH and fails to be degraded, so that it exhibits behavior as a constitutive active mutant (Non-patent document 13).

It has been reported that ROS are involved in stabilization of HIF-1α. It has been found that generation of ROS in the mitochondria increases under hypoxia conditions, contributing to stabilization of HIF-1α (Non-patent document 14). In the presence of a mitochondrial respiratory chain inhibitor, HIF-1 activity was inhibited by allowing an inhibitor that suppresses ROS production to act on cells, while conversely HIF-1 activity was increased by allowing an inhibitor that promotes ROS production to act on cells, even under normal oxygen conditions (Non-patent document 14). In mitochondrial DNA-deficient cells, HIF-1 activation did not occur even under hypoxia conditions. HIF-1 was activated upon cellular overexpression of SOD, which increases hydrogen peroxide production. These results indicate that ROS are involved in stabilization of HIF-1α.

Even under normal oxygen conditions, HIF-1α expression is induced by several growth factors (for example, insulin, insulin-like growth factors 1 and 2, epidermal growth factor, fibroblast growth factor 2, platelet-derived growth factor and transforming growth factor β1) and inflammatory cytokines (for example, IL1 and TNFα) (Non-patent document 11, 15).

HIF-1 has already been found to activate the transcription of numerous genes. For example, it activates the transcription of genes for vascular endothelial growth factor that promotes angiogenesis, erythropoietin that promotes erythrocyte production, glycolytic enzymes involved in energy metabolism (lactate dehydrogenase, aldolase A and the like), glucose transporter 1 and BNIP3 that promotes apoptosis. Most of these genes have been shown to contain the HIF-1-binding sequence 5′-(A/G)CGTG-3′ (Non-patent document 11).

As mentioned above, it is thought that mutant SOD1, NEDL1, ROS and inflammatory cytokines (IL1, TNFα, etc.) are closely involved in onset of FALS. However, it is thought that these molecules alone are insufficient for onset of FALS, and the existence of a molecule that connect these substances functionally has been predicted.
[Non-patent document 1] Rosen D R et al. Nature 364:59-62, 1993
[Non-patent document 2] Wong P C et al. Neuron 14:1105-1116, 1995
[Non-patent document 3] Liu D et al., FASEB J. 13: 2318-2328, 1999
[Non-patent document 4] Said Ahmed M et al., J Neurol Sci. 176: 88-94, 2000
[Non-patent document 5] Jung C et al., Neurosci Lett. 304: 157-160, 2001
[Non-patent document 6] Di Matteo V and Esposito E., Curr Drug Targets CNS Neurol Disord. 2: 95-107, 2003
[Non-patent document 7] Hensley K et al., J. Neurochem. 82: 365-374, 2002
[Non-patent document 8] Yoshihara T et al., J. Neurochem. 80: 158-167, 2002
[Non-patent document 9] Miyazaki K et al., J Biol. Chem. 279: 11327-11335, 2004
[Non-patent document 10] Wang G L and Semenza G L. J. Biol. Chem. 268: 21513-21518, 1993
[Non-patent document 11] Zagorska A and Dulak J. Acta Biochim. Pol. 51: 563-585, 2004
[Non-patent document 12] Wang G L and Semenza G L. Blood 82: 3610-3615, 1993
[Non-patent document 13] Yu F et al. Proc Natl Acad Sci USA. 98: 9630-9635, 2001
[Non-patent document 14] Chandel N S et al. Proc Natl Acad Sci USA. 95:11715-11720, 1998
[Non-patent document 15] T Hell-B et al., Blood 94:1561-1567, 1999

### DISCLOSURE OF THE INVENTION

### Problems to be Solved by the Invention

The discovery of a pathway that regulates expression of NEDL1 will lead to the development of therapeutic or prophylactic agents for FALS. It is therefore one object of the invention to provide a pathway that regulates expression of NEDL1. It is another object of the invention to provide a screening method for compounds that inhibit onset and progression of FALS. It is yet another object of the invention to provide a method for diagnosis of FALS.

### Means for Solving the Problems

The present inventors used a DNA chip (carrying approximately 11,000 neuroblastoma-derived genes) to study genes whose expression is altered under hypoxia conditions, in order to explore target genes of HIF-1 in human neuroblastoma, and have surprisingly found that expression of the NEDL1 gene is increased. As a result of further experimentation using RT-PCR methods and the like, it was found that NEDL1 expression is regulated downstream from HIF-1. The present inventors have completed the invention based on this knowledge.

Specifically, the invention provides a screening method for compounds that inhibit onset or progression of FALS, comprising a step of culturing cells in the presence and in the absence of a test compound; a step of measuring HIF-1α expression in the respective cultured cells, and a step of determining the test compound to be a compound that inhibits onset or progression of FALS if the HIF-1α expression in the cells cultured in the presence of the test compound is less than the HIF-1α expression in the cells cultured in the absence of the test compound. This screening method is based on a mechanism whereby NEDL1 expression is regulated downstream from HIF-1. The screening method allows compounds to be obtained which inhibit onset or progression of FALS by a different mechanism than currently known. The compounds may be applied as therapeutic or prophylactic agents for FALS.

The invention further provides a method for diagnosis of FALS comprising a step of measuring HIF-1α expression in neurons taken from a subject is measured, and a step of determining the subject to be suffered from FALS if the HIF-1α expression is high. This diagnosis method is based on a mechanism whereby NEDL1 expression is regulated downstream from HIF-1. It allows diagnosis of FALS to be determined by different criteria than currently known.

### EFFECT OF THE INVENTION

The screening method of the invention allows compounds to be obtained which inhibit onset or progression of FALS by a different mechanism than currently known. The method for diagnosis of FALS according to the invention allows diagnosis of FALS to be determined by different criteria than currently known and permits diagnosis of FALS at an early stage.

### BRIEF DESCRIPTION OF THE DRAWINGS

FIG. 1 is a photograph showing NEDL1 mRNA expression in SK-N-BE (2) C cells cultured under hypoxia conditions (2% oxygen).

FIG. 2 is a photograph showing NEDL1 mRNA expression in SK-N-BE (2) C cells cultured under different conditions.

FIG. 3 is a photograph showing NEDL1 mRNA expression in SK-N-BE (2) C cells expressing wild-type and constitutively activated HIF-1α.

FIG. 4 is a schematic diagram showing the relationship between HIF-1 and NEDL1.

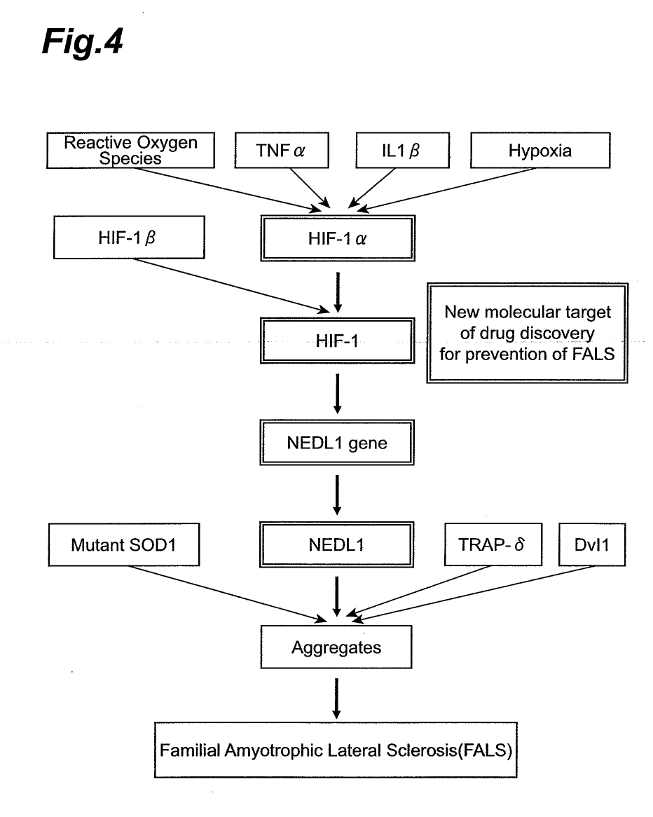

### BEST MODE FOR CARRYING OUT THE INVENTION

Preferred embodiments of the invention will now be described in detail.

(Screening Method for Compounds that Inhibit Onset or Progression of FALS)

The screening method for compounds that inhibit onset or progression of FALS comprises a step of culturing cells in the presence and in the absence of a test compound; a step of measuring HIF-1α expression in the respective cultured cells; and a step of determining the test compound to be a compound that inhibits onset or progression of FALS if the HIF-1α expression in the cells cultured in the presence of the test compound is less than the HIF-1α expression in the cells cultured in the absence of the test compound.

The cells used for the screening method are not particularly restricted so long as they are cells that express HIF-1α, and SK-N-BE (2) C human neuroblastoma cells may be mentioned as an example. The cells are cultured both in the presence and in the absence of the test compound.

The HIF-1α expression in the respective cultured cells is then measured. Here, the HIF-1α expression is HIF-1α mRNA and/or HIF-1α protein expression. Measurement of the HIF-1α expression may be accomplished by a method known to those skilled in the art. For example, mRNA may be measured by Northern blotting, quantitative or semi-quantitative RT-PCR, RNase protection assay or the like. Protein may be measured by Western blotting or the like.

If the measurement results indicate that HIF-1α expression in the cells cultured in the presence of the test compound is lower than HIF-1α expression in the cells cultured in the absence of the test compound, then the test compound may be determined to be a compound that inhibits onset or progression of FALS.

(FALS Diagnosis Method)

The method for diagnosis of FALS according to the invention comprises a step of measuring HIF-1α expression in neurons taken from a subject, and a step of determining the subject to be suffered from FALS if the HIF-1α expression is high.

Neurons are taken from the subject by a method known to those skilled in the art. Expression of HIF-1α is then measured by a method for measuring HIF-1α expression as described above, and FALS determined based on the level of expression. For example, a subject may be determined to be suffered from FALS if HIF-1α expression in the subject is above a prescribed level with respect to the level of HIF-1α expression in neurons taken from a healthy person.

### EXAMPLES

The present invention will now be explained in greater detail with reference to examples, with the understanding that the invention is not meant to be limited to these examples.

(Construction of HIF-1α Expression Vector)

Human HIF-1α cDNA (GenBank Accession No.: NM_{—}001530) cloned in pBluescript KS(−) was obtained from Dr. Gassmann. HIF-1α cDNA was cut from the plasmid using EcoRV and NotI, electrophoresed on agarose gel and then purified using a Gel Extraction Kit (Qiagen Inc.). It was then subcloned at the EcoRV/NotI site of pcDNA3.1(+) (Invitrogen Corp.) to construct plasmid pcDNA3/HIF-1α.

HIF-1α^{P402A/P564A} having the 402nd and 564th prolines (P) substituted with alanine (A) was prepared in the following manner. First, a Quick Change Site-Directed Mutagenesis Kit (Stratagene) and mutagenesis primers 5′-CTTGGAGATGTTAGCTGCCTATATCCCAATGGATGA-3′ (SEQ ID NO: 1) and 5′-TCATCCATTGGGATATAGGCAGCTAACATCTCCAAG-3′(SEQ ID NO: 2) were used according to the manufacturer's manual, to construct pcDNA3/HIF-1α^{P564A} having the 564th proline of pcDNA3/HIF-1α substituted with alanine. Next, pcDNA3/HIF-1α^{P402A/P564A} having the 402nd proline of pcDNA3/HIF-1α^{P564A} substituted with alanine was constructed in the same manner using mutagenesis primers 5′-CTTTGCTGGCCGCAGCCGCTGGAG-3′(SEQ ID NO: 3) and 5′-CTCCAGCGGCTGCGGCCAGCAAAG-3′(SEQ ID NO: 4).

(Cell Culturing)

Human neuroblastoma SK-N-BE (2) C cells were cultured in Dulbecco's Minimum Essential Medium (DMEM) containing 10% heat treated fetal bovine serum (FBS). Hypoxia culturing was carried out in a 2% oxygen concentration, with the cells in a Napco automatic O₂/CO₂ cell culture apparatus (Waken Co., Ltd.). DFO (Sigma) treatment was carried out by exposing the cells to 250 μM DFO for 24 hours.

(Gene Transfection)

Gene transfection into the cells was accomplished using a Lipofectamine 2000 (Invitrogen Corp.) by the following procedure. The cells were seeded on a 6 cm diameter culture dish (#3002, Falcon) at approximately 70% confluence at the time of gene transfection, and were cultured overnight at 37° C. After adding 4 μL of Lipofectamine 2000 to 100 μL of Opti-MEM medium (Invitrogen Corp.) in a polystyrene tube (#2058, Falcon), the mixture was allowed to stand at room temperature for 5 minutes. To this there was added 2.5 μg of plasmid DNA (pcDNA3.1, pcDNA3/HIF-1α or pcDNA3/HIF-1α^{P402A/P564A}) dissolved in 100 μL of Opti-MEM medium, and the mixture was allowed to stand at room temperature for 15 minutes to form DNA-liposome complexes. The cell medium was exchanged with 3 mL of Opti-MEM medium, and the DNA-liposome complexes were added to cover the entire culture dish. After culturing at 37° C. for 5 hours, 0.6 mL of FBS and 2.4 mL of Opti-MEM medium were added. On the following day, the medium was exchanged with 10% FBS-containing DMEM and culturing was continued for 48 hours.

(Reverse Transcription Polymerase Chain Reaction (RT-PCR)) TRIZOL reagent (Invitrogen Corp.) was used according to the manufacturer's manual to prepare total RNA from untreated, hypoxia-treated, DFO-treated cells and the transfected cells. RT-PCR was conducted in the following manner. The prepared total RNA (1 μg/2.5 μL) was incubated at 75° C. for 5 minutes and then allowed to stand on ice for 5 minutes. The prepared RNA was added to a mixture of 0.5 μL RNase inhibitor (40 units/μL, Toyobo, Ltd.), 0.5 μL M-MLV reverse transcriptase (200 units/μL, Invitrogen Corp.), 1 μL 50×dNTPs (2 mM each), 0.5 μL oligo(dT) primer (0.5 mg/mL, Invitrogen Corp.), 2 μL 5×first-strand buffer (Invitrogen Corp.) and 1 μL 0.1 M dithiothreitol, and the mixture was incubated for 10 minutes at room temperature, 45 minutes at 37° C. and 3 minutes at 95° C. to prepare cDNA. The prepared cDNA was used as template with the primers under the following PCR conditions to amplify the NEDL1 and β-actin gene cDNA fragments. After mixing 1 μL cDNA, 2 μL 5×buffer, 0.5 μL dNTPs (2 mM each), 0.05 μL GoTaqDNA polymerase (5 units/mL, Promega) and 0.5 μL each of sense and antisense primer (10 μM), water was added to a total volume of 10 μL. PCR was carried out using a GeneAmp PCR System 9700 (Applied Biosystems). As the PCR conditions, first heat denaturation was carried out at 95° C. for 2 minutes, and was followed by 32-35 cycles of heat denaturation (95° C., 15 sec), annealing (60° C., 15 sec) and extension (72° C., 20 sec), and then by incubation at 72° C. for 6 minutes and cooling to 10° C. The nucleotide sequences of the primers used were the following.

NEDL1 sense primer: 5′-CCAGGGAACCAAAGCATAGA-3′ (SEQ ID NO: 5)
Antisense primer: 5′-GTGGATGCGGATGTAGTGCG-3′ (SEQ ID NO: 6)
β-Actin sense primer: 5′-TGACGGGGTCACCCACACTGTGCCCATCTA-3′ (SEQ ID NO: 7)
Antisense primer: 5′-CTAGAAGCATTTGCGGTGGACGATGGAGGG-3′ (SEQ ID NO: 8)

The amplified PCR products were electrophoresed on 1% agarose gel containing ethidium bromide and the DNA bands were observed in an UV illuminator.

### Example 1

DNA chip analysis of the genes with altered expression, using the total RNA prepared from SK-N-BE (2) C cells cultured for 24 hours under normal oxygen and hypoxia (2% oxygen) conditions, indicated increased NEDL1 gene expression (data not shown).

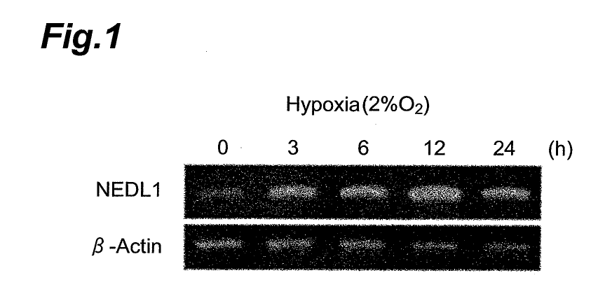

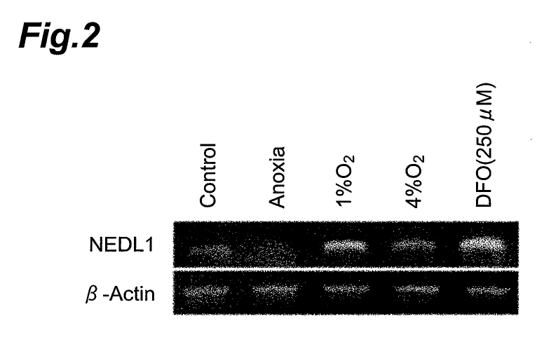

Total RNA was then prepared from SK-N-BE (2) C cells cultured for 0, 3, 6, 12 and 24 hours in a 2% oxygen concentration, and examination of NEDL1 mRNA expression by semi-quantitative RT-PCR confirmed increased NEDL1 mRNA expression in response to hypoxia (FIG. 1). NEDL1 mRNA expression was also increased with 1% oxygen conditions and DFO treatment (FIG. 2).

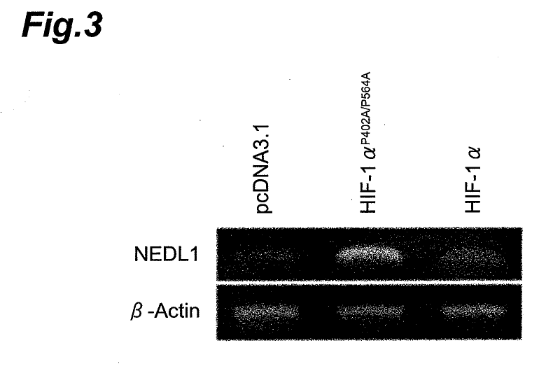

In order to examine whether the increased NEDL1 mRNA expression was mediated by HIF-1, the wild-type or constitutively activated HIF-1α-expressing vector pcDNA3/HIF1-α or pcDNA3/HIF1-α^{P402A/P564A} was transfected into the cells and expression of the NEDL1 gene was examined. The results showed a clear increase in NEDL1 mRNA expression in the pcDNA3/HIF1-α^{P564A/P564A} transfected cells (FIG. 3). No increase in expression was seen in the pcDNA3/HIF-1α transfected cells, and this was attributed to degradation of the wild-type HIF-1α.

### INDUSTRIAL APPLICABILITY

The screening method of the invention allows compounds to be obtained which inhibit onset or progression of FALS by a different mechanism than currently known. The method for diagnosis of FALS according to the invention allows diagnosis of FALS to be determined by different criteria than currently known and permits diagnosis of FALS at an early stage.

1. A method for screening compounds that inhibit onset or progression of familial amyotrophic lateral sclerosis, comprising
a step of culturing cells in the presence and in the absence of a test compound,
a step of measuring HIF-1α expression in the respective cultured cells, and
a step of determining the test compound to be a compound that inhibits onset or progression of familial amyotrophic lateral sclerosis if the HIF-1α expression in the cells cultured in the presence of the test compound is less than the HIF-1α expression in the cells cultured in the absence of the test compound.

2. A method for diagnosis of familial amyotrophic lateral sclerosis, comprising
a step of measuring HIF-L a expression in neurons taken from a subject, and
a step of determining the subject to be suffered from familial amyotrophic lateral sclerosis if the HIF-1α expression is high.
